(19) United States
(12) Patent Application Publication
(10) Pub. No.: US 2021/0087274 A1
(43) Pub. Date: Mar. 25, 2021

(54) APPLICATION OF PLANT AS HOST IN EXPRESSING PD-1 ANTIBODY AND/OR PD-L1 ANTIBODY

(71) Applicant: Kevin WANG, West Linn, OR (US)

(72) Inventors: Kevin WANG, West Linn, OR (US); Wen LI, Wuhan, Hubei (CN); Shunchang JIAO, Beijing (CN)

(73) Assignee: Kevin WANG, West Linn, OR (US)

(21) Appl. No.: 16/621,771

(22) PCT Filed: Jul. 18, 2017
(86) PCT No.: PCT/CN2017/093244

§ 371 Date: Dec. 12, 2019

(30) Foreign Application Priority Data
Jun. 16, 2017 (CN) 201710458315.4

(51) Int. Cl. C07K 16/28; C12N 15/82

(52) U.S. Cl. CPC C07K 16/2818; C12N 15/8205; C07K 16/2827

(57) ABSTRACT

Provided is an application of a plant as a host in expressing a PD-1 antibody and/or a PD-L1 antibody, wherein the plant, such as lettuce, is used as an effective expression platform for preparing recombinant proteins, and a simple and effective agrobacterium-mediated vacuum infiltration method is used for expressing the PD-1 monoclonal antibody (Keytruda, pembrolizumab) and the PD-L1 monoclonal antibody (Atezolizumab).

The present application claims priority of Chinese Patent Application No. 201710458315.4, entitled “APPLICATION OF PLANT AS HOST IN EXPRESSING PD-1 ANTIBODY AND/OR PD-L1 ANTIBODY”, filed on Jun. 16, 2017 at the Chinese Patent Office, the entire contents of which are incorporated herein by reference.

### FIELD OF THE INVENTION

The present invention relates to the field of biotechnology, and in particular to use of a plant as a host for expressing a PD-1 antibody and/or a PD-L1 antibody.

### BACKGROUND OF THE INVENTION

Cancer is the leading cause of death worldwide, and its incidence is rising due to population growth and aging, as well as other ubiquitous factors such as pollution in air and foods. In the treatment, surgery, chemotherapy and radiotherapy are still the main means of treating various types and stages of tumors at this stage. However, due to the lack of selectivity for tumor cells, the chemotherapy methods has limited success rate and can lead to systemic toxicity and drug resistance. Radiotherapy cannot kill all cancer cells and may cause the patient's body to become weaker. Because cancer cells are diffuse, surgery can remove the site of the disease, but cannot block the spread of cancer cells. At present, more advanced therapies are based on the molecular characteristics of tumor cells to design better targeted therapies to prevent their growth and spread. Most of these therapies are based on small molecule drugs that readily enter tumor cells or monoclonal antibodies (mAbs) that bind to specific targets on their surface.

MAb-based targeted therapies are immunotherapies for different targets, such as by blocking the oncogenic pathway, affecting cell growth and apoptosis, blocking new blood vessel formation, regulating immune responses to tumor cells, regulating osteoclast function or delivering cytotoxic drugs to kill tumor cells. Since the Food and Drug Administration (FDA) approved the first monoclonal antibody (Rituximab®), hundreds of antibodies, including murine, chimeric and humanized antibodies, have been developed for cancer treatment. Some of these monoclonal antibodies have been approved by the FDA and are already available for clinical applications in daily practice as monotherapy or in combination with standard chemotherapy regimens, while many other monoclonal antibodies are still being tested in different clinical trials.

The PD-1 antibody (Pembrolizumab, anti-PD-1, Keytruda®) is a humanized monoclonal antibody that binds to the PD-1 receptor and blocks its interaction with PD-L1 and PD-L2 ligands. Keytruda® was first approved by the FDA in 2014 for the treatment of patients with unresectable or metastatic melanoma. In 2015, Keytruda® received approvals for two other indications; in October, are used to treat patients with metastatic NSCLC expressing PD-L1 in platinum chemotherapy or subsequent treatment, and in December, are used to treat patients with unresectable or metastatic melanoma. Recently in October 2016, the FDA approved an accelerated approval procedure based on tumor response rate and tolerability for the treatment of patients with recurrent or metastatic head and neck squamous cell carcinoma (HNSCC) in platinum chemotherapy or subsequent treatment. The PD-1 antibody (Keytruda) was approved in March 2017 for the treatment of Hodgkin's lymphoma (cHL).

PD-L1 antibody (Anti-PD-L1, Atezolizumab, Tecentriq™) is an Fc engineered humanized monoclonal antibody that binds to PD-L1 and blocks its interaction with the PD-1 and B7.1 receptors. It addresses the inhibition of PD-L1/PD-1 mediated immune responses, including activation of anti-tumor immune responses without induction of antibody-dependent cellular cytotoxicity. Anti-PD-1L (Atezolizumab) was approved by the FDA in 2016 for the treatment of patients with locally advanced or metastatic urothelial carcinoma, and has a therapeutic effect on metastatic cancer cells during chemotherapy and can be used for adjuvant therapy within 12 months after chemotherapy. In October 2016, Atezolizumab was approved by the FDA for use in patients with metastatic non-small cell lung cancer (NSCSC) who were treated for BGFR or ALK genomic aberrations during or after chemotherapy.

Because PD-1 and PD-L1 antibodies have significant killing effect on cancer cells, they can significantly improve the survival rate of cancer patients. Because of its broad-spectrum anti-cancer effect, the FDA is also accelerating the clinical phase II or III trial of the antibody against cancers such as kidney cancer, stomach cancer, breast cancer, bladder cancer, blood cancer, head and neck cancer, colon cancer and brain tumors. At present, PD-1 and PD-L1 antibodies are produced by mammalian cells, but their low yield, complex processing, and high equipment requirements make it difficult to meet the high demand of cancer patients.

Currently, animal cells are used to produce PD-1 and PD-L1 monoclonal antibodies. However, the culture of animal cell requires expensive culture medium, strict conditions, complicated operations, a period of at least two weeks, and extremely high costs due to the low production of animal cell. Sometimes the virus carried by animal cells may infect humans, resulting in low safety of the product.

### SUMMARY OF THE INVENTION

Therefore, the present invention provides use of a plant as a host for expressing a PD-1 antibody and/or a PD-L1 antibody. The present invention utilizes plants, especially lettuce, as a highly efficient platform for the production of recombinant proteins, such as PD-1 and PD-L1 antibodies. Furthermore, exogenous proteins with activity were successfully isolated under mild conditions, demonstrating that plants, especially lettuce expression system, can be successfully used to produce PD-1 and PD-L1 antibodies. The advantages of this method include short production period (4 d), simple purification, convenient operation, less genetic pollution, less potential pests for humans, etc., greatly reducing production costs and improving product safety.

In order to achieve the above object, the present invention provides the following technical solutions.

The present invention provides use of a plant as a host for expressing a PD-1 antibody and/or a PD-L1 antibody. Preferably, the antibody is a monoclonal antibody.

In some specific embodiments of the invention, the plant is selected from the group consisting of lettuce, tobacco, Chinese cabbage, rice, corn, soybean and wheat; and the organ of the plant is selected from the group consisting of a leaf, a seed, a rhizome and a whole plant.

The invention also provides an expression vector comprising a vector and any one of:

i. a heavy chain sequence or a light chain sequence of PD-1;

ii. a heavy chain sequence or a light chain sequence of PD-L1.

In some specific embodiments of the invention, the codons of the heavy chain sequence or the light chain sequence of PD-1 are optimized to plant-preferred codons to obtain an optimized PD-1 heavy chain sequence or an optimized PD-1 light chain sequence; and

the codons of the heavy chain sequence or the light chain sequence of PD-L1 are optimized to plant-preferred codons to obtain an optimized PD-L1 heavy chain sequence or an optimized PD-L1 light chain sequence.

In some specific embodiments of the invention, the optimized PD-1 heavy chain sequence is set forth in SEQ ID No: 1; the nucleotide sequence of the optimized PD-1 heavy chain is set forth in SEQ ID No: 2.

The optimized PD-1 light chain sequence is set forth in SEQ ID No: 3; the nucleotide sequence of the optimized PD-1 light chain sequence is set forth in SEQ ID No: 4.

The optimized PD-L1 heavy chain sequence is set forth in SEQ ID No: 5; the nucleotide sequence of the optimized PD-L1 heavy chain is set forth in SEQ ID No: 6.

The optimized PD-L1 light chain sequence is set forth in SEQ ID No: 7; and the nucleotide sequence of the optimized PD-L1 light chain is set forth in SEQ ID No: 8.

In some specific embodiments of the invention, the vector is a binary plant vector.

In some specific embodiments of the invention, the method of constructing the expression vector comprises the steps of:

Step 1: respectively optimizing the codons of the PD-1 heavy chain, the PD-1 light chain, the PD-L1 heavy chain and the PD-L1 light chain to plant-preferred codons to obtain

i. optimized PD-1 heavy chain sequence,

ii. optimized PD-1 light chain sequence,

iii. optimized PD-L1 heavy chain sequence, and

iv. optimized PD-L1 light chain sequence;

Step 2: respectively adding XbaI restriction site at the 5′ end and XhoI site at the 3′ end of the optimized PD-1 heavy chain sequence, the optimized PD-L1 heavy chain sequence and the optimized PD-L1 light chain sequence;

adding XmaI restriction site at the 5′ end and XhoI site at the 3′ end of the optimized PD-1 light chain sequence;

respectively cloning the sequences into pUC57 vector by genecript to obtain pPD-1H, pPD-1L, pPD-L1H and pPD-L1L cloning vectors; and

Step 3: producing gene fragments from the cloning vectors obtained in Step 2 by KpnI/SacI, and cloning the gene fragments into the binary plant vector pCam35S to obtain expression vectors p35S-PD-1H, p35S-PD-1L, p35S-PD-L1H and p35S-PD-L1L.

Specifically, in order to provide high-efficiency expression of exogenous proteins in plants, the codons of the human PD-1 heavy chain (GenBank Accession number: 5DK3_B) and light chain, (GenBank Accession number: 5DK3_A), PD-L1 heavy chain (GenBank Accession No.: AAO 17823.1) and light chain (GenBank Accession number: 4DKE_L) are optimized to plant-preferred codons using the protein sequence reverse translation software (https://www.idtdna.com/CodonOpt). The optimized sequences are synthesized by Genescript (Nanjing, China). XbaI restriction site is added at the 5′ end and XhoI site is added at the 3′ end of the optimized PD-1 heavy chain sequence, as well as PD-L1 light and heavy chain sequence. XmaI restriction site is added at the 5′ end and XhoI site is added at the 3′ end of the PD-1 light chain sequence, respectively. The sequences are cloned into pUC57 vector by genecript, to obtain pPD-1H, pPD-1L, pPD-L1H and pPD-L1L cloning vectors, respectively. The gene fragments are generated from the cloning vectors by KpnI/SacI digestion, and cloned into the binary plant vector pCam35S to obtain expression vectors p35S-PD-1H, p35S-PD-1L, p35S-PD-L1H and p35S-PD-L1L, respectively.

The invention also provides use of the expression vector for expressing a PD-1 antibody and/or a PD-L1 antibody.

In addition, the present invention also provides a method for expressing PD-1 antibody and/or a PD-L1 antibody using a plant as a host by transforming the expression vector provided by the present invention into agrobacterium, performing agrobacterium-mediated vacuum infiltration on a plant tissue, and then extracting and isolating the protein to obtain the PD-1 antibody and/or the PD-L1 antibody.

Specifically, four plant expression vectors p35S-PD-1H, p35S-PD-1L, p35S-PD-L1H and p35S-PD-L1L are transformed into Agrobacterium tumefaciens GV3101 by electroporation using Multiporator (Eppendorf, Hamburg, Germany), respectively. The resulting strains are spread evenly on selective LB plates containing kanamycin (50 mg/L). After incubating at 28° C. in the dark for 2 days, single colonies are picked and inoculated into 0.5 L YEB liquid medium (yeast extract broth, 5 g/L sucrose, 5 g/L tryptone, 6 g/L yeast extract, 0.24 g/LMgSO₄, pH 7.2) supplemented with antibiotic (50 mg/L kanamycin). The inoculated culture is incubated at 25 to 28° C. in a shaker (220 rpm) for 72 h. The O.D.600 value is measured and adjusted to 3.5 to 4.5 by adding YEB medium. The culture medium is then collected and centrifuged (4,500 rpm) for 10 min. The agrobacterium cells are resuspended in penetrating solution (10 mM MES, 10 mM MgSO4) until O.D.600 is 0.5.

The agrobacterium containing p35S-PD-1H and the agrobacterium containing p35S-PD-1L are mixed in equal amounts to an O.D.600 of 0.5; also, the agrobacterium containing p35S-PD-L1H and the agrobacterium containing p35S-PD-L1L are mixed in equal amounts to an O.D.600 of 0.5. Each mixed suspension is added into a 2 L beaker and the beaker is placed in a desiccator. The lettuce leaves are inverted (core up) and gently spun in the bacterial suspension, and the desiccator is then sealed. Vacuum is applied using a vacuum pump (Welch Vacuum, Niles, Ill., USA) and the penetrating solution in the leaf tissue is observed. After keeping under the pressure for 30˜60 s, the pressure is released quickly, allowing the penetrating solution to penetrate into the space inside the tissue. This procedure is repeated 2 to 3 times until the significant diffusion of penetrating solution in the lettuce tissue is clearly visible. The lettuce tissue is then gently removed from the penetrating solution and rinsed three times with distilled water and then transferred to a container covered with a plastic film. The treated samples are kept in the dark for 4 days.

In some specific embodiments of the invention, the agrobacterium-mediated vacuum infiltration comprises the steps of:

Step 1: vacuuming for 25˜45 s;

Step 2: maintaining under a vacuum of −95 kPa for 30˜60 s;

Step 3: releasing the pressure and allowing penetrating solution to penetrate into the plant tissue; and

repeating Step 1 to Step 3 for 2 to 3 times, and then keeping the plant tissue in the dark for 4 d.

In some specific embodiments of the invention, the agrobacterium is Agrobacterium tumefaciens GV3101.

Using pPD-1H, pPD-1L, pPD-L1H and pPD-L1L gene fragments, four binary plant expression vectors p35S-PD-1H, p35S-PD-1L, P35S-PD-L1H and p35S-PD-L1L are constructed according to FIGS. 2A and 2B. After completion of the construction, digestion with specific restriction enzymes is carried out to confirm the intact gene fragment. During vacuum infiltration, most of the lettuce tissues are submerged in the penetrating solution. Except for the solid midrib area, the remaining parts of the lettuce turn to yellowish brown 4 days after vacuum infiltration.

The extraction and separation of proteins are specifically as follows: The lettuce sample after agrobacterium vacuum infiltrated is stirred with a stirrer and homogenized at a high speed in the extraction buffer (100 mM KPi, pH 7.8; 5 mM EDTA; 10 mM β-mercaptoethanol) at 1:1 ratio for 1 to 2 minutes. The homogenate is adjusted to pH 8.0, filtered through gauze, and the filtrate is centrifuged at 10,000 g for 15 min at 4° C. to remove cell debris. The supernatant is collected, mixed with ammonium sulfate (50%), and incubated on ice for 60 min with shaking, and separated again by centrifuge (10,000 g) at 4° C. for 15 min. The resulting supernatant is subjected to a second round of ammonium citrate (70%) precipitation, incubated on ice for 60 min with shaking, and centrifuged again at 10,000 g for 15 min at 4° C. Then, the supernatant is discarded, and the precipitated protein from the treated sample is dissolved in 5 mL buffer (20 mM KPi, pH 7.8; 2 mM EDTA; 10 mM β-mercaptoethanol) and stored at 4° C.

SDS-PAGE gel electrophoresis is performed as follows: the proteins extracted from the lettuce after agrobacterium vacuum infiltrated are collected. 5 μL sample is heat-denatured (95° C.), mixed with loading buffer (Biorad, Hercules, Calif., USA), and subjected to electrophoresis on 4 to 12% Bolt® Bis-Tris Plus SDS-gel (ThermoFisher Scientific, Waltham, Mass., USA). Also, the affinity of the antibody is detected by non-denaturing gel electrophoresis. After staining with Coomassie Blue G250 (Biorad), the gel is photographed again.

Downstream processing of plant-derived recombinant protein is often difficult and expensive because cellulose cell walls are difficult to lyse and secondary plant metabolites are produced. Stirrer is used to perform homogenization, which greatly saves the cost and process of homogenization. After recombinant PD-1 antibody and PD-L1 antibody are separated by denaturing SDS-PAGE gel, bands with estimated molecular weights of approximately 23 kDa and 50 kDa are observed (FIG. 3A), which are consistent with the protein sizes of the light chain and heavy chain of PD-1 and PD-L1 antibodies. A band of approximately 150 kDa (FIG. 3B) is observed in non-denaturing gel electrophoresis, consistent with the protein molecular weight of PD-1 and PD-L1 antibodies, demonstrating successful forming of antibody structure by the light chain and heavy chain in lettuce. The protein content in the purified sample is determined to be approximately 0.72 mg/g based on the Bradford assay and densitometric control group.

The obtained antibody is verified by a cancer cell inhibition test. Cells of human non-small cell lung cancer (NSCLC) cell line A549 are grown in RPMI-1640 medium (Gibco, USA) supplemented with 10% FBS (Gibco, USA), 100 U/mL penicillin and 100 μg/mL streptomycin. All cells are cultured in 5% CO₂ humidified atmosphere at 37° C., the medium is changed daily, and the cells are passaged every three days using 0.25% trypsin. A549 cells are collected by trypsinization and resuspended at a density of 1×10⁶ cells/mL. The purified 10 μg of PD-1 and PD-L1 antibodies are added respectively, and double staining is performed with annexin V-luciferin isothiocyanate (FITC) and PI to evaluate the proportion of apoptotic cells.

Inhibition of human non-small cell lung cancer (NSCLC) cells by purified PD-1 and PD-L1 antibodies is investigated by cell experiments. After the purified recombinant PD-1 and PD-L1 antibodies are added to the cultured NSCLC cells and the cell growth is checked at 72 h. The results show that the NSCLC cells without treatment grow well, in contrast, cells incubated with purified recombinant PD-1 and PD-L1 antibodies are mostly destroyed (FIG. 4). These results indicate that exogenous PD-1 and PDL-1 antibodies transiently expressed by the lettuce system are biologically active and can kill NSCLC cells. The results indicate that plants, especially lettuce, are a suitable bioreactor for the production of PD-1 and PD-L1 antibodies.

The present invention utilizes lettuce to transiently express PD-1 and PD-L1 antibodies, which gives high levels of protein in a relatively short period (4 d). Lettuce is a higher plant that has a post-translational modification process, so that the expressed protein may have activity automatically. This approach minimizes biosafety issues because processed lettuce tissue is usually developed in fully enclosed facilities or containers without biological pollution problems. Lettuce basically does not contain plant toxic substances, and contains less fiber, which is beneficial to downstream protein purification. The use of lettuce systems to produce PD-1 and PD-L1 monoclonal antibodies can significantly reduce production time and costs.

### BRIEF DESCRIPTION OF THE FIGURE

In order to more clearly illustrate the embodiments of the present invention or the technical solutions in the prior art, the drawings to be used in the embodiments or the description of the prior art will be briefly described below.

FIG. 1 shows a map of the cloning vector pUC57.

FIG. 2 (A) shows the construction of the PD-1 plant binary expression vector p35S-PD-1H (heavy chain) and P35S-PD-1L (light chain). PD-1H heavy chain sequence is obtained by double digestion with restriction endonuclease (Xbal/XhoI) from the cloning vector of FIG. 1 and inserted into the Xbal/XhoI sites of pCam35S to generate the plant binary expression vector P35S-PD-1H; the PD-1H heavy chain sequence is obtained by double digestion with restriction endonuclease (Xma1/XhoI) from the cloning vector of FIG. 1 and inserted into the Xma1/XhoI sites of pCam35S to generate the plant binary expression vector P35S-PD-1L;

LB and RB: the left and right borders of the Ti plasmid; 35S, the CaMV 35S promoter with the tobacco mosaic virus (TMV) 5′UTR; NPT II, the expression of the nptII gene for kanamycin resistance; Nos3′, Terminator.

FIG. 2 (B) shows the construction of the PD-L1 plant binary expression vector p35S-PD-L1H (heavy chain) and P35S-PD-L1L (light chain). PD-L1H light chain and PD-L1L heavy chain sequences are obtained by double digestion with restriction endonuclease (Xbal/XhoI) from the cloning vectors of FIG. 1 and inserted into Xbal/XhoI sites of pCam35S to generate the plant binary expression vector P35S-PD-L1H and P35S-PD-L1L;

LB and RB: the left and right borders of the Ti plasmid; 35S, the CaMV 35S promoter with the tobacco mosaic virus (TMV) 5′UTR; NPT II, the expression of the nptII gene for kanamycin resistance; Nos3′, Terminator.

FIG. 3 (A) shows the results of SDS-PAGE gel electrophoresis; Lane 1: PD-1 recombinant antibody; Lane 2: PD-L1 recombinant antibody;

FIG. 3 (B) shows the results of non-denaturing gel electrophoresis; Lane 3: PD-1 recombinant antibody; Lane 4: PD-L1 recombinant antibody;

FIG. 4 shows the inhibition of human non-small cell lung cancer (NSCLC) cells by purified PD-1 and PD-L1 antibodies in cell experiments.

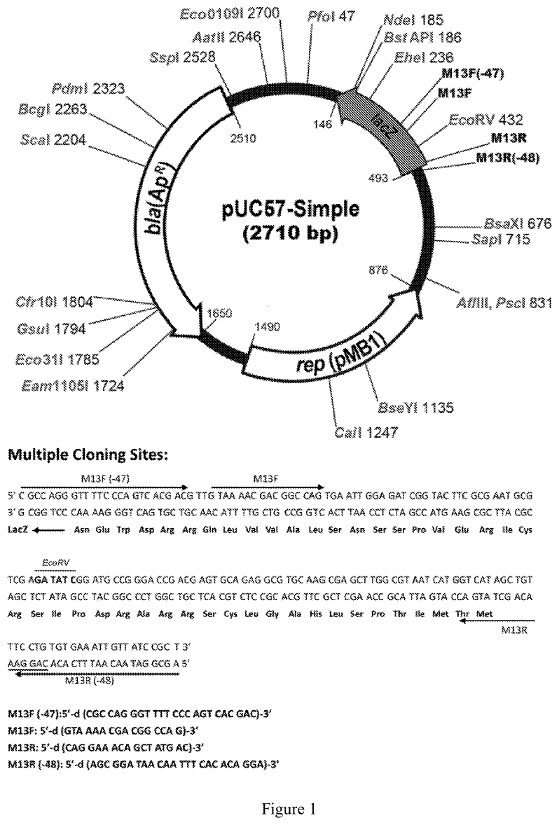

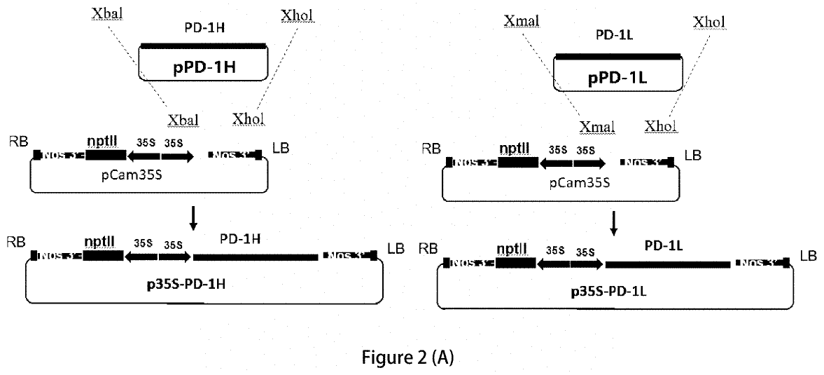

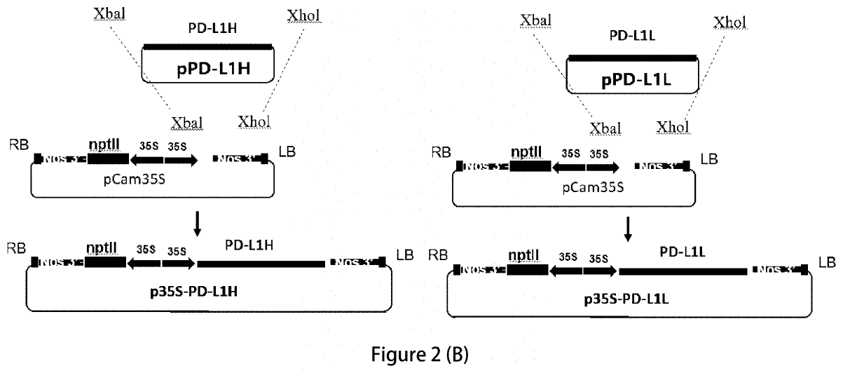

### DETAILED DESCRIPTION OF THE INVENTION

The invention discloses use of a plant as a host for expressing a PD-1 antibody and/or a PD-L1 antibody. Those skilled in the art can learn from the contents of this document and appropriately improve the process parameters. It is specifically to be understood that all such alternatives and modifications are obvious to those skilled in the art and are considered to be included in the present invention. The methods and applications of the present invention have been described by the preferred embodiments, and it is obvious that the methods and applications described herein can be modified or appropriately changed and combined to implement and apply the techniques of the present invention without departing from the scope of the present invention.

The present inventor has found through experiments that a plant system, especially a lettuce system, is a more economical and efficient expression platform for transient expression of recombinant proteins. The vacuum agrobacterium infiltration method described in the present invention is a simple and rapid method which can increase recombinant protein production. Lettuce can increase protein production by withstanding vacuum pressure and allow for a more complete penetration of each leaf. Since lettuce is easy to grow and commercially mass-produced, it is easier to be obtained and cheaper than other transiently expressed plants, such as tobacco, and the cost can be significantly reduced since no complicated special production equipment is required. In summary, the present invention can utilize the lettuce system to mass produce PD-1 and PD-L1 monoclonal antibodies in a short period of time.

The materials and reagents used in the application of the plant as a host for expressing PD-1 antibody and/or a PD-L1 antibody according to the present invention are commercially available.

The present invention is further illustrated below in conjunction with the examples.

### Example 1. Construction of Plant Transient Expression Vector

In order to provide high-efficiency expression of exogenous proteins in plants, the codons of the human PD-1 heavy chain (GenBank Accession number: 5DK3_B) and light chain, (GenBank Accession number: 5DK3_A), PD-L1 heavy strand (GenBank Accession No.: AAO 17823.1) and light chain (GenBank Accession number: 4DKE_L) are optimized into plant-preferred codons using the protein sequence reverse translation software (https://www.idtdna.com/CodonOpt) and synthesized by Genescript (Nanjing, China). XbaI restriction site was added at the 5′ end and XhoI site was added at the 3′ end of the optimized PD-1 heavy chain, as well as PD-L1 light chain and heavy chain sequences, respectively. XmaI restriction site was added at the 5′ end and XhoI site was added at the 3′ end of the PD-1 light chain sequence, respectively. The sequences were cloned into pUC57 vector by genecript, to obtain pPD-1H, pPD-1L, pPD-L1H and pPD-L1L cloning vectors, respectively. The gene fragments were cut from the cloning vector by KpnI/SacI digestion, and cloned into the binary plant vector pCam35S to obtain expression vectors p35S-PD-1H, p35S-PD-1L, p35S-PD-L1H and p35S-PD-L1L, respectively. The four plant expression vectors were transformed into Agrobacterium tumefaciens GV3101 by electroporation using Multiporator (Eppendorf, Hamburg, Germany), respectively. The resulting strains were spread evenly on selective LB plates containing kanamycin antibiotic (50 mg/L). After incubating at 28° C. in the dark for 2 days, single colonies were picked and inoculated into 0.5 L YEB liquid medium (yeast extract broth, 5 g/L sucrose, 5 g/L tryptone, 6 g/L yeast extract, 0.24 g/LMgSO₄, pH 7.2) supplemented with antibiotic (50 mg/L kanamycin). The inoculated culture was incubated at 25 to 28° C. for 72 h in a shaker (220 rpm). The O.D.600 value was measured and adjusted to 3.5 to 4.5 by adding YEB medium. The culture medium was then collected and centrifuged (4500 rpm) for 10 min. The agrobacterium cells were resuspended in penetrating solution (10 mM MES, 10 mM MgSO4) until O.D.600 was 0.5.

The obtained pPD-1H, pPD-1L, pPD-L1H and pPD-L1L gene fragments were cloned, and four binary plant expression vectors p35S-PD-1H, p35S-PD-1L, P35S-PD-L1H and p35S-PD-L1L were constructed. After completion of the construction, digestion with specific restriction enzymes was performed to confirm that the gene fragment was intact. During vacuum infiltration, most of the lettuce tissue was submerged in the penetrating solution, and except for the solid midrib area, the remaining parts of lettuce turned yellowish brown 4 days after vacuum infiltration.

### Example 2. Agrobacterium-Mediated Vacuum Infiltration

The prepared agrobacterium containing p35S-PD-1H and agrobacterium containing p35S-PD-1L were mixed in equal amounts to O.D.600 of 0.5; also, the prepared agrobacterium containing p35S-PD-L1H and agrobacterium containing p35S-PD-L1L were mixed in equal amounts to O.D.600 of 0.5. The culture suspension was added into a 2 L beaker and the beaker was placed in a desiccator. The lettuce was inverted (core up) and gently spun in the bacterial suspension, and the desiccator was then sealed. Vacuum was applied using a vacuum pump (Welch Vacuum, Niles, Ill., USA) and the penetrating solution in the leaf tissue was observed. After keeping under the pressure for 30˜60 s, the pressure was released quickly, allowing the penetrating solution to penetrate into the space inside the tissue. This procedure was repeated 2 to 3 times until the significant diffusion of penetrating solution in the lettuce tissue was clearly visible. The lettuce tissue was then gently removed from the penetrating solution and rinsed three times with distilled water and then transferred to a container covered with a plastic film. The treated samples were kept in the dark for 4 days.

### Example 3 Protein Extraction and Isolation

The lettuce sample after agrobacterium vacuum infiltrated was stirred in a stirrer and homogenized at a high speed in the extraction buffer (100 mM KPi, pH 7.8; 5 mM EDTA; 10 mM β-mercaptoethanol) at 1:1 ratio for 1 to 2 minutes. The homogenate was adjusted to pH 8.0, filtered through gauze, and the filtrate was centrifuged at 10,000 g for 15 min at 4° C. to remove cell debris. The supernatant was collected, mixed with ammonium sulfate (50%), and incubated on ice for 60 min with shaking, and then was again separated by a centrifuge (10,000 g) at 4° C. for 15 min. The resulting supernatant was subjected to a second round of ammonium citrate (70%) precipitation, suspended on ice for 60 min with shaking, and again centrifuged at 10,000 g for 15 min at 4° C. Then, the supernatant was discarded, and the precipitated protein from the treated sample was dissolved in 5 mL buffer (20 mM KPi, pH 7.8; 2 mM EDTA; 10 mM β-mercaptoethanol) and stored at 4° C.

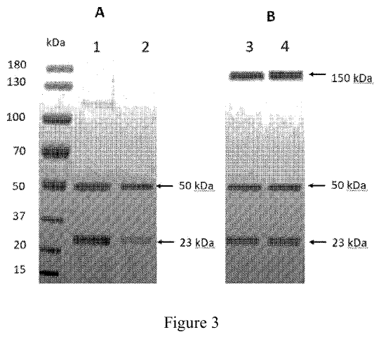

Downstream processing of plant-derived recombinant proteins is often difficult and expensive because cellulose cell walls are difficult to lyse and secondary plant metabolites are produced. In the present invention, a stirrer is used to perform homogenization, which greatly saves the cost and process of homogenization. After recombinant PD-1 antibody and PD-L1 antibody were separated by denaturing gel SDS-PAGE, bands with estimated molecular weights of approximately 23 kDa and 50 kDa in the lanes were observed (FIG. 3A), consistent with the protein sizes of the light and heavy chains of PD-1 and PD-L1 antibodies. A band of approximately 150 kDa (FIG. 3B) was observed in non-denaturing gel electrophoresis, consistent with the protein molecular weight of PD-1 and PD-L1 antibodies, demonstrating successful formation of the antibody structure by the light chain and heavy chain in lettuce. The protein content in the purified sample was determined to be approximately 0.72 mg/g based on the Bradford assay and the densitometric control group.

### Example 4 SDS-PAGE Gel Electrophoresis

The purified protein extracted from the lettuce after agrobacterium vacuum infiltrated was collected, and the sample (5 μL) was heat-denatured (95° C.), mixed with loading buffer (Biorad, Hercules, Calif., USA), and then electrophoresed on 4 to 12% Bolt® Bis-Tris Plus SDS-gel (ThermoFisher Scientific, Waltham, Mass., USA). Also, the affinity of the antibody was detected in non-denaturing gel electrophoresis. After staining with Coomassie Blue G250 (Biorad), the gel was photographed again.

### Example 5. Inhibition Experiment on Cancer Cells

Cells of human non-small cell lung cancer NSCLC cell line A549 were grown in RPMI-1640 medium (Gibco, USA) supplemented with 10% FBS (Gibco, USA), 100 U/mL penicillin and 100 μg/mL streptomycin. All cells were cultured in a 5% CO₂ humidified atmosphere at 37° C., with the medium changed daily, and cells were passaged every three days using 0.25% trypsin. A549 cells were collected by trypsinization and resuspended at a density of 1×10⁶ cells/mL. 10 μg of purified PD-1 and PD-L1 antibodies were added, respectively, and double staining was performed with annexin V-luciferin isothiocyanate (FITC) and PI to evaluate the proportion of apoptotic cells.

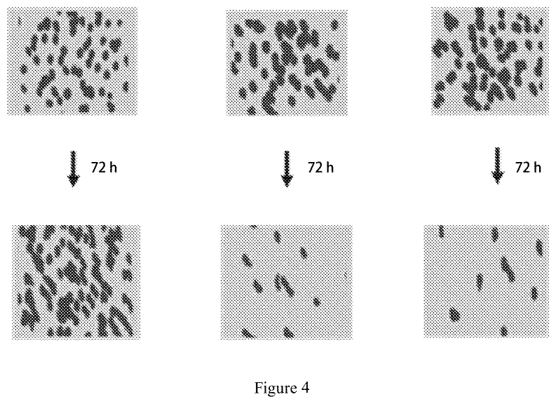

The results show inhibition of lung cancer (NSCLC) cells. The purified recombinant PD-1 and PD-L1 antibodies were added to the cultured NSCLC cells, and the cell growth was checked at 72 h. The results showed that the NSCLC cells without treatment grew well, in contrast, cells cultured with purified recombinant PD-1 or PD-L1 antibodies mostly were destroyed (FIG. 4). These results indicate that exogenous PD-1 and PDL-1 antibodies transiently expressed by the lettuce system are biologically active and can kill NSCLC cells. The results indicate that plants, especially lettuce, are a suitable bioreactor for the production of PD-1 and PD-L1 antibodies.

### Example 6

Control group: PD-1 and PD-L1 antibodies produced in animals;

Experimental group 1: PD-1 and PD-L1 antibodies produced in the plant provided by the present invention;

Experimental group 2: PD-1 and PD-L1 antibodies produced in leaves of tobacco.

TABLE 1
PD-1 and PD-L1 antibodies

| | | | | The degree of difficulty | |
|---|---|---|---|---|---|
| | Production | Protein | Protein | in the downstream | Production |
| Group | cycle (d) | content | activity | protein purification | cost (yuan) |
| Control | 14 | 0.42 | Binding assay | It is difficult to remove | About 16000 |
| group | | mg/g | 1.21 × 10⁶ | animal cell impurities. In | yuan per |
| | | | | particular, animal cells | gram of |
| | | | | are often contaminated | protein |
| | | | | with human viruses and | |
| | | | | have low safety. | |
| Experimental | 4**^{#} | 0.59**^{#} | Binding assay | Relatively easy. The | About |
| group 1 | | mg/g | 1.82 × 10⁶**^{#} | downstream homogenization | 6500**^{##} yuan |
| | | | | is carried out with a stirrer, | per gram of |
| | | | | saving time and money, | protein |
| | | | | and eliminating the need | |
| | | | | to remove impurities such | |
| | | | | as nicotine and nicotine. | |
| Experimental | 7* | 0.51* | Binding assay | Relatively difficult. The | About |
| group 1 | | mg/g | 1.27 × 10⁶* | time-consuming, | 13400* yuan |
| | | | | laborious and expensive | per gram of |
| | | | | liquid nitrogen grinding | protein |
| | | | | was required, and special | |
| | | | | steps were required to | |
| | | | | remove nicotine, nicotine | |
| | | | | impurities. | |

*indicates P ≤ 0.05 compared with the control group;
**indicates P ≤ 0.01 compared with the control group;
^{#}indicates P ≤ 0.05 compared with the experimental group 2;
^{##}indicates P ≤ 0.01 compared with the experimental group 2.

As can be seen from Table 1, compared with the animal expression system of the control group, a lettuce transient expression system is provided by the present invention for the expression of PD-1 and PD-L1 antibodies, which reduces the production cycle very significantly (P≤0.01), improves the protein content very significantly (P≤0.01), improves the protein activity significantly (P≤0.05), simplifies the purification of the protein and reduces production costs very significantly (P≤0.01).

Compared with the tobacco expression system of experimental group 2, the lettuce transient expression system for PD-1 and PD-L1 antibodies reduces the production cycle significantly (P≤0.05), improves the protein content significantly (P≤0.05), improves the protein activity significantly (P≤0.05), simplifies the purification of the protein and reduces production costs very significantly (P≤0.01).

Compared with the control group, the tobacco leaf transient expression system of experimental group 2 for PD-1 and PD-L1 antibodies reduces the production cycle significantly (P≤0.05), improves the protein content significantly (P≤0.05), improves the protein activity significantly (P≤0.05), simplifies the purification of the protein and reduces production costs significantly (P≤0.05).

The above test results show that plant system, especially lettuce system, is a more economical and efficient expression platform for rapid and transient expression of recombinant proteins, and can produce large-scale PD-1 and PD-L1 monoclonal antibodies in a short period of time.

The above application of a plant as a host for expressing a PD-1 antibody and/or a PD-L1 antibody according to the present invention is described in detail. The principles and embodiments of the present invention are set forth herein in terms of specific examples, and the description of the above embodiments is only to aid in understanding the method of the present invention and its core concepts. It should be noted that those skilled in the art can make various modifications and changes to the present invention without departing from the spirit and scope of the invention.

1. A method for expressing a PD-1 antibody and/or a PD-L1 antibody, comprising using a plant as a host.

2. The method according to claim 1, wherein the plant is selected from the group consisting of lettuce, tobacco, Chinese cabbage, rice, corn, soybean and wheat, and the organ of the plant is selected from the group consisting of a leaf, a seed, a rhizome and a whole plant.

3. An expression vector comprising a vector and
i. a heavy chain sequence or a light chain sequence of PD-1; or
ii. a heavy chain sequence or a light chain sequence of PD-L1.

4. The expression vector according to claim 3, wherein
the codons of the heavy chain sequence or the light chain sequence of PD-1 are optimized to plant-preferred codons to obtain an optimized PD-1 heavy chain sequence or an optimized PD-1 light chain sequence; and
the codons of the heavy chain sequence or the light chain sequence of PD-L1 are optimized to plant-preferred codons to obtain an optimized PD-L1 heavy chain sequence or an optimized PD-L1 light chain sequence.

5. The expression vector according to claim 4, wherein
the optimized PD-1 heavy chain sequence is set forth in SEQ ID No: 1, the nucleotide sequence of the optimized PD-1 heavy chain is set forth in SEQ ID No: 2;
the optimized PD-1 light chain sequence is set forth in SEQ ID No: 3, the nucleotide sequence of the optimized PD-1 light chain sequence is set forth in SEQ ID No: 4;
the optimized PD-L1 heavy chain sequence is set forth in SEQ ID No: 5, the nucleotide sequence of the optimized PD-L1 heavy chain is set forth in SEQ ID No: 6; and
the optimized PD-L1 light chain sequence is set forth in SEQ ID No: 7, the nucleotide sequence of the optimized PD-L1 light chain is set forth in SEQ ID No: 8.

6. The expression vector according to claim 3, wherein the vector is a binary plant vector.

7. The expression vector according to claim 3, which is constructed by the steps of:
Step 1: respectively optimizing the codons of the PD-1 heavy chain, the PD-1 light chain, the PD-L1 heavy chain and the PD-L1 light chain to plant-preferred codons to obtain
i. optimized PD-1 heavy chain sequence,
ii. optimized PD-1 light chain sequence,
iii. optimized PD-L1 heavy chain sequence, and
iv. optimized PD-L1 light chain sequence;
Step 2: respectively adding XbaI restriction site at the 5′ end and XhoI site at the 3′ end of the optimized PD-1 heavy chain sequence, the optimized PD-L1 heavy chain sequence and the optimized PD-L1 light chain sequence;
adding XmaI restriction site at the 5′ end and XhoI site at the 3′ end of the optimized PD-1 light chain sequence;
respectively cloning the sequences into pUC57 vector to obtain pPD-1H, pPD-1L, pPD-L1H and pPD-L1L cloning vectors; and
Step 3: producing gene fragments from the cloning vectors obtained in Step 2 by KpnI/SacI, and cloning the gene fragments into the binary plant vector pCam35S to obtain expression vectors p35S-PD-1H, p35S-PD-1L, p35S-PD-L1H and p35S-PD-L1L.

8. A method for expressing a PD-1 antibody and/or a PD-L1 antibody comprising using the expression vector according to claim 3.

9. The according to claim 8, comprising
transforming the expression vector into agrobacterium,
performing agrobacterium-mediated vacuum infiltration on a plant tissue, and
extracting and isolating protein to obtain the PD-1 antibody and/or the PD-L1 antibody.

10. The method according to claim 9, wherein the agrobacterium-mediated vacuum infiltration is performed by:
Step 1: vacuuming for 25˜45 s;
Step 2: maintaining under a vacuum of −95 kPa for 30˜60 s;
Step 3: releasing the pressure and allowing penetrating fluid to penetrate into the plant tissue; and
repeating Step 1 to Step 3 for 2 to 3 times, and then keeping the plant tissue in the dark for 4 d.


SEQUENCE LISTING (8 sequences)

SEQ ID NO: 1 - DNA, 1341 bp, Artificial
  PD-1 heavy chain nucleotide sequence
caagtccagctggtacaaagtggcgtggaagttaaaaaaccgggggcttcagtgaaagtgagttgcaaagccagcggttatacttttacaaattactatatgtactgggttagacaagcccctggacagggccttgaatggatgggaggaatcaacccgtctaatggtgggactaatttcaacgaaaaattcaaaaatagagttaccctcacgactgattcatcaacaacgactgcctacatggaacttaaaagcctccaatttgatgacacagcggtatattactgcgctcgacgagactacagatttgacatgggattcgattactgggggcagggtaccaccgtcacagtttctagtgctagcacgaagggcccctcagtcttcccacttgcgccttgctccagaagcacatccgagagcaccgcagcactgggctgtctcgtaaaggattatttcccggagcctgttactgtctcatggaacagcggtgcgttaaccagtggcgtgcacacttttccagcagtgctccaatcatctgggctttactcacttagctctgtcgtgaccgtaccgtccagttctcttggcactaagacatatacgtgtaatgtcgatcacaaaccttcaaacacgaaagtcgacaaacgagtcgaatctaagtacggtccaccgtgcccaccctgtccagccccagaattcctaggggggccgtccgtatttttattccctcccaagcccaaagacacattgatgatatcacgaacaccggaagtcacgtgcgtagtggtcgacgtgtcccaggaggatcctgaagttcaatttaactggtatgtggacggagtggaagtacataatgcaaaaactaagcctagggaggagcagtttaatagcacatacagggtagtatccgtgctaactgtgttgcaccaagattggttgaacggtaaagagtataagtgcaaagtatccaataaagggttgccgagctccattgagaaaactattagtaaagcaaagggacaaccgagagaaccgcaagtctataccttaccgccgagccaggaggagatgaccaaaaatcaagtatcattgacgtgtcttgttaaaggcttttatccctcagacattgccgtcgaatgggaatctaacggacaaccggagaataattataagactacgccacctgtgttggactctgatggtagtttcttcttatattcccgtctgaccgttgataagtcaagatggcaggaaggcaatgtgttctcctgtagtgttatgcacgaagcactacacaaccactacacccaaaagtccctatctttgtcactgggaaaa

SEQ ID NO: 2 - protein, 447 aa, Artificial
  PD-1 heavy chain amino acid sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 654 bp, Artificial
  PD-1 light chain nucleotide sequence
gaaatcgtgctaactcaaagcccggcgacgctctcattaagccctggagaacgagccaccttgtcatgtcgtgcgtctaaaggggtgtccacgagcggctatagctatctacactggtaccagcaaaagccgggacaggccccccgactattgatctatttggcatcttatctagagtctggtgttcctgcgcgattttccgggagcgggagtggaacagactttacgctgaccatcagcagtctggagccggaagacttcgcagtctactattgtcagcattctagggatctccccctaacatttggcggtggtactaaagttgaaatcaagcgtactgttgccgcaccaagtgtctttatctttcccccttctgacgaacaactaaaaagcggtaccgcgagtgtggtatgtctcttgaacaacttttatccgcgagaggcgaaagtccagtggaaagtcgataatgctctgcaatccggcaactcacaggagtccgtaactgagcaagactctaaggatagtacctacagcttgtcaagtacattgactctatctaaagctgactatgaaaagcataaagtgtacgcctgcgaagttactcaccaagggttgagcagtccggtaacgaagtcattcaatagaggggagtgt

SEQ ID NO: 4 - protein, 218 aa, Artificial
  PD-1 light chain amino acid sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1344 bp, Artificial
  PD-L1 heavy chain nucleotide sequence
gaggtgcagctagtggagtccggtggaggacttgtccagcccggtggaagcttaagactatcttgtgctgcatctgggttcactttttctgattcctggattcactgggtacgacaggcgcctggcaagggcctagaatgggtagcatggatctcaccgtatggcggctctacatactacgctgacagcgttaaaggtcgatttacgatctctgctgacacatccaagaacacagcctatctccagatgaatagtcttagagctgaagataccgcagtttattactgtgctagaagacactggcctggcggcttcgattattgggggcagggcaccctagtcacagtttccagtgcctccactaaaggccccagcgtctttccgttagcaccttcctcaaagagcactagcggagggactgccgctctcgggtgtctggtcaaggactacttcccggagccagtcacagtgtcttggaattcaggggccctcacctcaggtgtccacacttttccggcggtgctgcaaagttctggcctatactccctgtctagtgtcgttacggtcccgagcagctctctcggcacgcaaacgtatatatgcaatgtcaaccataagccgtcaaataccaaagttgataagaaggtggagccgaaaagctgtgataagactcatacatgcccaccatgcccggcacctgagttgctgggaggaccgagtgtattcttatttccgcccaagcccaaggacactctaatgattagtagaactccagaggttacatgtgtagtggtagatgtttcacacgaagatccggaagtgaagttcaactggtacgtggacggggtggaggtacacaatgcgaaaactaaaccccgtgaagaacagtacgcttctacttacagagttgtctcagtgctaaccgtactgcatcaagactggttgaacggcaaggagtataagtgtaaagtctccaataaggccttgccggcgcccatagaaaaaaccatctccaaagcaaaagggcagccgcgagagcctcaagtttataccttaccaccttctagggaagaaatgacgaagaatcaagtgtctctcacatgcttagtcaagggattctatcctagtgatattgccgttgaatgggaatctaacggacaaccggaaaataattataaaaccacgccgcctgtcttagactctgacgggtccttcttcctttatagcaaacttactgtcgacaagtcacgatggcaacagggaaacgtgttttcttgctcagtaatgcacgaagcgctccataatcactatacgcaaaagtctttgagcctctctccagggaag

SEQ ID NO: 6 - protein, 448 aa, Artificial
  PD-L1 heavy chain amino acid sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 642 bp, Artificial
  PD-L1 light chain nucleotide sequence
gacatacaaatgacacaatccccatccagcttgtccgccagtgtcggggatcgagttacaatcacctgccgagctagtcaagatgttagtaccgccgtagcctggtaccaacaaaagcctgggaaggctccaaagctgctgatatatagtgcctcctttctatactctggtgtgcctagccgattttcaggatctgggagcggaaccgactttacgctcactatatcttccttgcaaccagaagatttcgccacgtattattgtcagcagtacctgtaccatcctgctactttcggtcagggcacgaaggtcgaaatcaagcgaacggtagcagctcccagcgtgttcatattcccaccttctgacgaacagctcaagtctggcactgcgagcgtagtttgcctcttaaataatttttatccccgtgaagcgaaagtgcaatggaaggtggataatgcattgcagtcaggtaactcacaggaatcagtaacggagcaggattcaaaggacagtacctattctttgagttcaaccttgacgttgtctaaagcggactacgaaaaacacaaggtttatgcgtgtgaggtcacgcaccagggactctcctcccctgtaacgaaatcatttaatagaggggaatgt

SEQ ID NO: 8 - protein, 214 aa, Artificial
  PD-L1 light chain amino acid sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC